(19) United States
(12) Patent Application Publication
(10) Pub. No.: US 2014/0038275 A1
(43) Pub. Date: Feb. 6, 2014

(54) Pharmacology Bioassays for Drug Discovery, Toxicity Evaluation and in vitro Cancer Research Using a 3D Nanocellulose Scaffold and Living Tissue

(71) Applicant: Paul Gatenholm, Riner, VA (US)

(72) Inventor: Paul Gatenholm, Riner, VA (US)

(21) Appl. No.: 13/989,686

(22) PCT Filed: Nov. 25, 2011
(86) PCT No.: PCT/US2011/062157

§ 371 Date: Oct. 24, 2013

Related U.S. Application Data
(60) Provisional application No. 61/416,917, filed on Nov. 24, 2010.
(60) Provisional application No. 61/439,636, filed on Feb. 4, 2011.
(60) Provisional application No. 61/552,376, filed on Oct. 27, 2011.

(51) Int. Cl. G01N 33/50

(52) U.S. Cl. CPC G01N 33/5088
USPC 435/288.4

(57) ABSTRACT

The present invention relates to pharmacology bioassays used in drug discovery, drug screening and toxicity evaluations. More specifically, the present invention relates to novel systems and methods used for production and control of 3-D architecture and morphology of living tissues and organs produced by mammalian cells using 3D porous scaffolds based on nano-cellulose. The resultant nano-cellulose based structures are useful as tools in high throughput assays for drugs. More particularly, embodiments of the present invention relate to systems and methods for evaluating a drug that comprise a microtiter plate comprising a plurality of wells, each well comprising: a 3D non-biodegradable, inert, nano-cellulose scaffold; and optionally cells capable of forming living tissue or organs; and optionally a drug having a biological activity of interest; and optionally a detector capable of detecting the biological activity in a high throughput format.

### CROSS-REFERENCE TO RELATED APPLICATIONS

The present application claims priority to and the benefit of the filing date of U.S. Provisional Application Nos. 61/416,917, filed Nov. 24, 2010; 61/552,376, filed Oct. 27, 2011; and 61/439,636, filed Feb. 4, 2011, the disclosures of which are hereby incorporated by reference herein in their entireties.

### BACKGROUND OF THE INVENTION

1. Field of the Invention

The present invention relates to pharmacology bioassays used in drug discovery, drug screening and toxicity evaluations. More specifically, the present invention relates to novel devices, systems and methods employing a plurality of engineered tissues and/or organs having a desired 3-D architecture and morphology supported by a 3D microporous nano-cellulose-based non-biodegradable scaffold, which can be used for high throughput drug discovery, screening, and toxicity testing. It can also be used to grow an artificial tumor and thus can be used for in vitro cancer research.

2. Discussion of Related Art

Cell-based assays play important roles in pharmacology. Currently typical cell assays are based on a 2D monolayer of cell culture on a microtiter plate and have been used extensively to assay for new drugs and screen the efficacy and toxicity of current drugs. The biological function of cells in a 2D monolayer of cell culture does not truly represent cells found in tissues and organs. Therefore, although these in vitro cell experiments may provide advantages for early screening of toxicity of new drugs by saving time and costs, the in vivo animal studies and clinical studies are still dominating the drug discovery process. Unfortunately, these animal and clinical studies are associated with many regulations, high costs, and a low throughput. There is a clear need for more efficient assays that would allow for high throughput pharmacologic screening of living tissues and organs in an in vitro assay.

Three-dimensional bioassay systems are distinguished over 2D systems in the superior distribution of oxygen, nutrients, metabolites, and signaling molecules, which are necessary to support cells. See Minchinton A I, Tannock I F, Drug penetration in solid tumours. Nat Rev Cancer 6:583-592 (2006); Malda J, Klein T J, Upton Z, The roles of hypoxia in the In vitro engineering of tissues. Tissue Eng Part A 13:2153-2162 (2007). Two-dimensional bioassay systems do not have the structure necessary to mimic living tissues and thus support cell life for sustained periods of time. See Yamada K M, Cukierman E,_{—}Modeling tissue morphogenesis and cancer in 3D. Cell 130:601-610 (2007); Schmeichel K L, Bissell M J, Modeling tissue-specific signaling and organ function in three dimensions. J Cell Sci 116:2377-2388 (2003); Pampaloni F, Reynaud E G, Stelzer E H K, The third dimension bridges the gap between cell culture and live tissue. Nat Rev Mol Cell Biol 8:839-845 (2007).

To fill this need, synthetic and natural based polymer 3D scaffolds with varying architecture have been developed in last decade to promote cell differentiation and production of extracellular matrix with the biochemical and biomechanical properties of native tissue in vitro. Most of the 3D scaffolds have been based on synthetic biodegradable polymers comprising polyglycolic acid or polylactic acid or their copolymers. Synthetic scaffolds with varying 3D architecture have been achieved by various production methods including fiber spinning, electrospinning, porogen introduction, free-form fabrication, freeze drying, etc. Such synthetic polymer based 3D scaffolds have been successfully used to grow a large variety of tissues and have been shown to promote stem cell differentiation into organs.

These biodegradable polymers, however, are designed to resorb into small molecular components during tissue growth. The typical degradation time for such biodegradable polymers varies from days to several weeks. This degradation process provides a great disadvantage in utilizing such 3D scaffolds for pharmacological bioassays. First, the biochemical composition of growing tissue may be strongly affected by chemicals derived from biodegradation of 3D scaffolds. Furthermore, the chemical signals derived from biodegradation of 3D synthetic scaffolds may disturb information required to measure drug efficacy and toxicity or other parameters in pharmacology applications.

Natural polymers have recently gained much attention as suitable candidates for 3D scaffolds for tissue growth and organ regeneration. Most recent applications are based on use of collagen derived from animal tissue. Collagen has been processed into foams, sponges or into nanofibril based materials using electrospinning technology. The use of collagen based 3D scaffolds has been quite successful for tissue engineering in academic research but the collagen source prevents its use as scaffold for humans in clinical applications. Particularly, the spreading of mad-cow disease has affected potential use of collagen as 3D scaffold. Another disadvantage of using collagen as scaffold in clinical application is the biochemical interactions collagen has with cells. For instance, the sequences of amino acids in the collagen structure are designed to act for cellular recognition and to promote cell adhesion and cell signaling thereby allowing collagen to be an active material. For use during the production of tissue, inert material would be much better. Furthermore collagen is biodegraded and biodegradation products may disturb the bioassays as discussed above.

Polysaccharides have been used instead of natural polymers such as collagens and elastins. Polysaccharides have less of an immunological impact and therefore have a more promising application in humans. Chitosan and hylauronic derivatives have been recently evaluated as candidates for 3D scaffolds for tissue engineering. Both polymers are however biodegradable in tissue growth environment and their degradation products may disturb the bioassays as discussed above. Thus, there is a clear need for 3D scaffolds that do not degrade and interfere with the sensitive bioassays.

To address the need that current 2D cell-culture systems do not accurately recapitulate the structure, function, or physiology of living tissues, simple systems have been developed. Cell cultures in stacked, paper-supported gels offer a uniquely flexible approach to study cell responses to 3D molecular gradients and to mimic tissue- and organ-level functions. Such systems were created to better replicate the spatial distributions of oxygen, metabolites, and signaling molecules found in tissues than which can be provided by existing 2D bioassay systems. Using stacking and destacking layers of paper impregnated with suspensions of cells in extracellular matrix hydrogel makes it possible to control oxygen and nutrient gradients in 3D and to analyze molecular and genetic responses. In the context of this technology, stacking assembles the “tissue”, whereas destacking disassembles it, and allows its analysis. It has been found that breast cancer cells cultured within stacks of layered paper recapitulate behaviors observed both in 3D tumor spheroids in vitro and in tumors in vivo: Proliferating cells in the stacks localize in an outer layer a few hundreds of microns thick, and growth-arrested, apoptotic, and necrotic cells concentrate in the hypoxic core where hypoxia-sensitive genes are overexpressed. Altering gas permeability at the ends of stacks controlled the gradient in the concentration of the O₂ and was sufficient by itself to determine the distribution of viable cells in 3D. R. Derda, A. Laromaine, A. Mammoto, S. Tang, T. Mammoto, D. Ingber, and G. Whitesides, Paper-supported 3D cell culture for tissue-based bioassays, Proceedings of the National Academy of Sciences of the United States of America, Sep. 17, 2009.

It is important to note that although existing techniques are approaching solutions to the deficiencies of 2D systems, the current solutions that polymers, existing microbially derived cellulose products, and other cellulose based products lack is the ability to control morphology of the structure during preparation or growth of the scaffold. As a consequence, because these techniques lack the requisite morphology, such scaffolds lack the ability to control or direct tissue integration into the implant once seeded with cells, and the ability to sustain life of the cells with vital fluids and nutrients. What is needed are biomimetic materials engineered with a structure that encourages tissue growth, i.e., a porous morphology having microporosity and/or macroporosity consistent with the natural tissue which it is intended to mimic.

### SUMMARY OF THE INVENTION

The numerous limitations inherent in known pharmacologic bioassays described above provide great incentive for new, better, and more efficient systems and methods capable of high throughput and cost-effective screening of drugs.

The primary limitation to the above-mentioned cell assays is their need to provide a 3D architecture similar to that found in living tissues and organs. However, the use of tissue engineering using 3D scaffolds provides the ability to reconstruct such tissues and organs in vitro. Unfortunately, the type of scaffold used may interfere with future pharmacologic bioassays or be inadequate to develop reliable models for living tissue or organs. Therefore, a more advanced system that allows for the production of a non-biodegradable, inert 3D scaffold from inexpensive and abundant materials, capable of growing living tissue, would be ideal.

Included in embodiments of the invention is a human liver bioassay system useful for determining toxicity of chemical agents and drugs in vitro. Currently, liver studies are mostly performed using hepatocytes cultured onto synthetic or animal-derived matrices. These models, however, fail to replicate true cell- matrix interactions found in vivo. In particular, the current 2D cell-based bioassays for studying drug metabolism and toxicity are limited because liver cells die rapidly in the 2D format. Human liver cells used for pre-clinical evaluation of drug metabolism profiles and hepatotoxicity testing typically lose function and die within hours in traditional cell culturing techniques. Due to phenotypic instability of isolated liver cells there is an urgent need for a long-term culture/assay model. The 3D scaffolds of the present invention are adapted for supporting long-term growth, survival and organization of many cell types, including human liver cells. Bioassay embodiments of the present invention include 3D or mini-liver assay platforms that exhibit functional benefits and allow precise toxicity prediction. High throughput adaptability of the bioassay systems according to the invention would allow parallel analysis of liver function, drug metabolism and toxicity profiling. If desired, the bioassay embodiments of the invention can be provided in the form of pre-packaged culture kits and coated plates and can serve as research and diagnostic tools.

Another application of embodiments of the invention is with respect to testing the in vivo effects of potential drug candidates on the heart. More particularly, mature human heart cells derived from pluripotent stem cells can be seeded onto scaffolds of the invention and used for screening the potential for heart toxicity or other non-toxic effects that drugs may have on the cardiovascular system.

Accordingly, some embodiments of the invention provide a system for evaluating a drug that comprises a microtiter plate comprising a plurality of wells, each well comprising: a 3D non-biodegradable, inert, porous nano-cellulose scaffold; cells capable of forming living tissue or organs; a drug having a biological activity of interest; and a detector capable of detecting the biological activity in a high throughput format. Embodiments of the invention include tools for bioassays having a microtiter plate with 3D non-biodegradable biosynthetic cellulose grown thereon in a desired 3D configuration, and optional features include one or more of the scaffold being inert, the scaffold having a desired amount of porosity, living cells seeded thereon, and/or one or more drug or gene as the feature to be tested using the assay.

What is meant by “porous” according to embodiments of the invention is that the particular nano-cellulose scaffold contains an amount of porosity, whether microporous or macroporous, to allow for nutrients, fluids, and other matter to be transported through the scaffold and to allow for the in-growth of cells into the scaffold. Such porosity is desired in order to mimic the morphology and function of natural tissue or organs. Preferably, porosity is introduced to the scaffold structure during the growth process of the bacterial cellulose. Alternatively or additionally, porosity may be introduced to the scaffold by mechanical means.

The term “inert” according to embodiments of the invention is that the scaffold does not contain substances that might tend to interfere with the assay being performed. Such substances can be removed from the scaffold using various techniques, including treating the scaffolds to NaOH wash for removal of unwanted substances, such as bacteria, that might be present after manufacture of the scaffolds.

Other embodiments of the present invention provide a kit for performing bioassays comprising: a microtiter plate comprising a plurality of wells, each well comprising a 3D non-biodegradable nano-cellulose scaffold with a target thickness and target porosity, such as macro-or micro-porosity. Optionally, such kits may also comprise living tissue or living cells for seeding the scaffolds contained in the microtiter plates.

Another embodiment of the present invention provides a method for evaluating the pharmacology of a drug comprising a microtiter plate comprising a plurality of wells, each well comprising: a 3D non-biodegradable, inert, porous nano-cellulose scaffold; cells capable of forming living tissue or organs; a drug having a pharmacological activity of interest; and a detector capable of detecting the pharmacologic activity in a high throughput format.

Yet another embodiment of the present invention provides a method for evaluating the toxicity of a drug comprising a microtiter plate comprising a plurality of wells, each well comprising: a 3D non-biodegradable, inert, porous nano-cellulose scaffold; cells capable of forming living tissue or organs; a drug having a toxicity; and a detector capable of detecting the toxicity in a high throughput format.

The features and advantages of the present invention will be apparent to those skilled in the art. While numerous changes may be made by those skilled in the art, such changes are within the spirit of the invention.

### BRIEF DESCRIPTION OF THE DRAWINGS

The accompanying drawings illustrate certain aspects of some of the embodiments of the present invention, and should not be used to limit or define the invention. Together with the written description the drawings serve to explain certain principles of the invention.

FIG. 1 is a Scanning Electron Micrograph (SEM) image of nano-cellulose material produced by the bacteria G. xylinus.

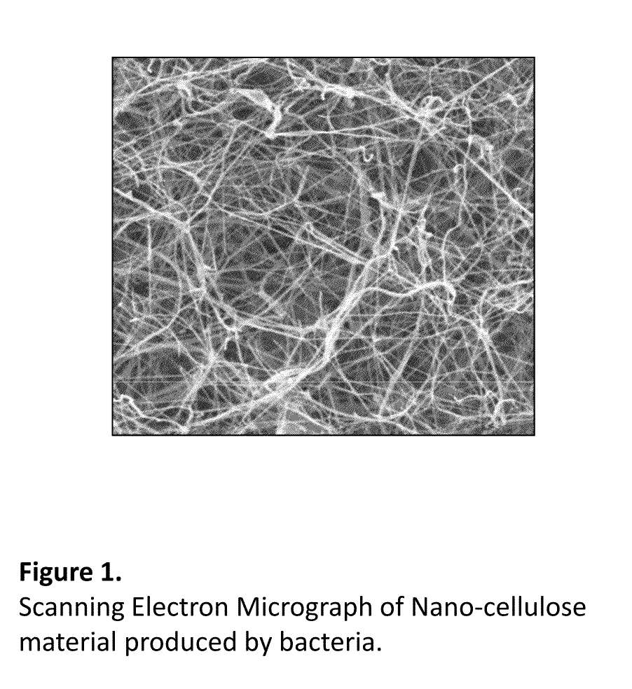

FIG. 2 is an SEM image of a 3D porous nano-cellulose scaffold capable of being used for tissue growth.

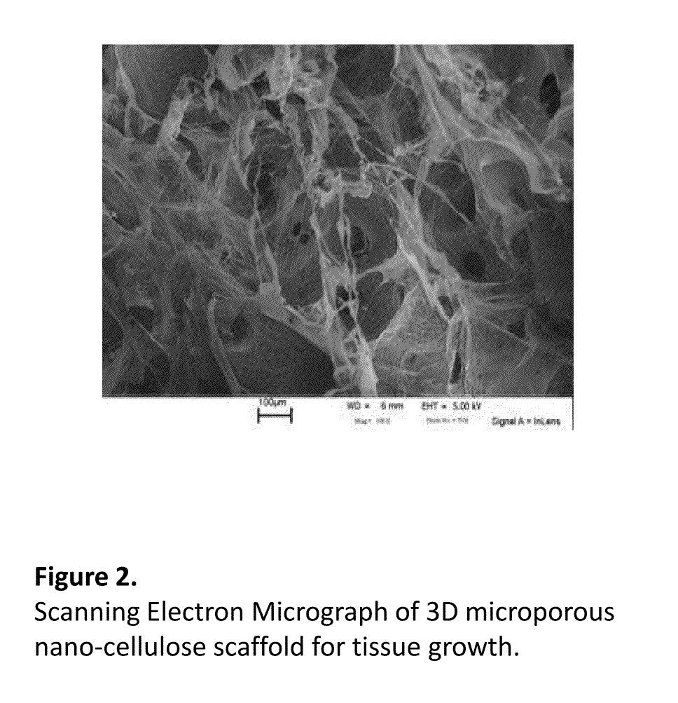

FIG. 3 is a schematic diagram illustrating how 3D porous nano-cellulose scaffold can be created using a 96-well microtiter plate and porous 3D bacterial cellulose.

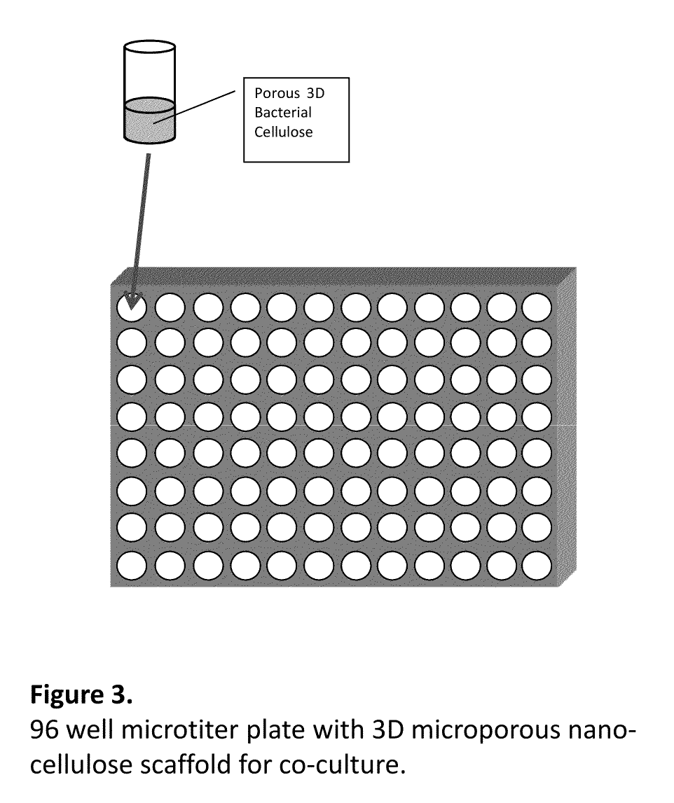

FIG. 4 is an image of Confocal scanning laser microscopy showing Osteoprogenitor cells that have migrated into a 3D porous nano-cellulose scaffold.

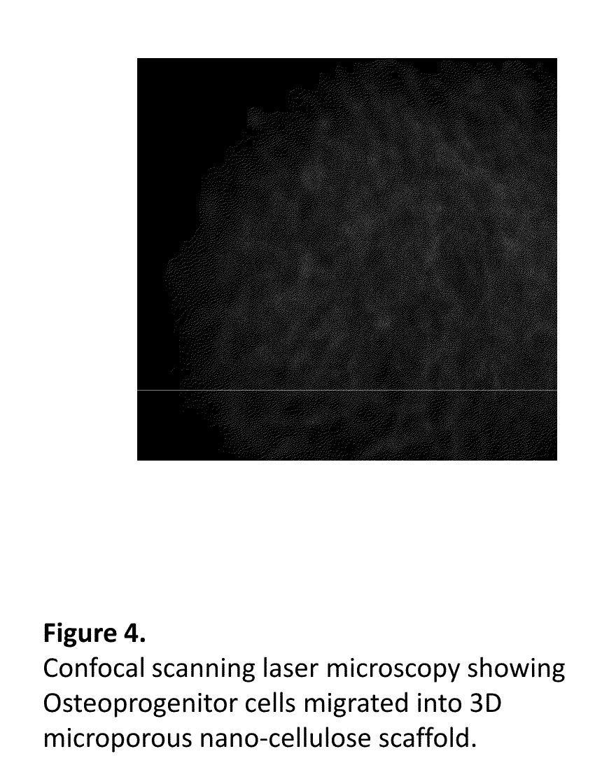

### DETAILED DESCRIPTION OF VARIOUS EMBODIMENTS OF THE INVENTION

Reference will now be made in detail to various exemplary embodiments of the invention. It is to be understood that the following discussion of exemplary embodiments is not intended as a limitation on the invention. Rather, the following discussion is provided to give the reader a more detailed understanding of certain aspects and features of the invention.

Embodiments of the invention include a device for high throughput bioassays comprising: a structural support having a plurality of recesses; and a plurality of non-biodegradable nano-cellulose scaffolds, each disposed in a recess of the support and having a selected 3D porous architecture. What is meant by “structural support” in this context is a substrate capable of supporting a plurality of tissue scaffolds in a manner which presents the tissue in ready form for in vitro testing. An example of a structural support that can be used according to embodiments of the invention is a microtiter plate with a plurality of wells in which the scaffolds can be disposed.

Such devices can comprise nano-cellulose which is fabricated in a non-mechanical manner to provide a network of biosynthetic cellulose, electrospun fibers or plant derived nanofibrilated cellulose. More particularly, the nano-cellulose material is preferable grown directly on the microtiter plates or can be grown in a bioreactor in a format that is compatible with placing the scaffolds once grown onto a microtiter plate. What is meant by “non-mechanical” in this context is that preferably the nano-cellulose material is not laminated with other sheets of nano-cellulose material to achieve a particular three-dimensional form. Rather, growth of the scaffold is controlled to obtain a scaffold grown into a desired 3D form and with a desired micro-porosity and macro-porosity needed for a particular application. Scaffolds prepared in this manner have a higher degree of similarity with the natural tissues which they are intended to resemble. Devices according to the invention further provide a 3D porous architecture that is selected to provide for cell differentiation and production of extracellular matrix.

The devices of embodiments of the invention can be seeded with living cells of any animal, including cells from humans; pigs, horses and other livestock; mice, etc. The cells can be differentiated stem cells or cells of particular organs or tissues of interest from any animal. Accordingly, the present invention has applications that extend into veterinary research as well.

In accordance with embodiments of the present invention, a method of the present invention may comprise providing a non-biodegradable, inert 3D scaffold for the production of living tissue and organs in vitro for use in a pharmacologic bioassay. In certain embodiments, the present invention relates to the use of a microtiter plate to perform high throughput assays for drug discovery, drug screening and drug toxicity and in vitro cancer research. In a preferred embodiment of the present invention, cellulose is used as the 3D scaffold.

Cellulose, a natural polysaccharide, is extremely attractive as a scaffold because of its good mechanical properties, hydroexpansivity, biocompatibility and its stability within a wide range of temperatures and pH levels. Cellulose ((β-1→4-glucan) is the most abundant polymer of natural origin. In addition to being biosynthesized in vast amounts as structural material in the walls of plants, cellulose is also produced as nanofibrils by bacteria, biosynthetic cellulose (BNC). Nano-cellulose can also be produced by electrospinning process or by isolation from annual plants or from wood. BNC has additional advantages as a scaffold as compared to plant-derived cellulose. For example, BNC has good mechanical strength, high water holding capacity, high purity and accessibility to non-aggregated micro fibrils, and can be biofabricated with control of porosity and microarchitecture that is crucial for cell differentiation. Another advantage of using nano-cellulose, only some of which are alluded herein, is that nano-cellulose is a non-biodegradable and inert scaffold, leading to controlled porosity and controlled scaffold-cell interactions.

Although BC scaffolds are preferred, any type of scaffold may be used according to the invention, including scaffolds derived from natural tissue sources. Especially preferred are natural tissue sources that have been treated to remove cellular debris while keeping the extracellular matrix in tact. For example, techniques of electroporation and/or enzyme treatments can be used on portions of natural human tissue to excise unwanted cell debris from the tissue, while leaving the extracellular matrix of the tissue unharmed. This provides for a scaffold with the desired porosity of the human tissue which it is intended to mimic.

Of particular interest are devices, systems, and methods of producing biosynthethic scaffolds disclosed in International Application No. PCT/US10/50460, filed Sep. 28, 2010 and entitled, “Three-Dimensional Bioprinting of Biosynthetic Cellulose (BC) Implants and Scaffolds for Tissue Engineering,” the disclosure of which is hereby incorporated by reference herein in its entirety. Provided in the disclosure is a method of producing 3-D nano-cellulose based structures comprising: providing bacteria capable of producing nano-cellulose; providing media capable of sustaining the bacteria for the production of nano-cellulose; controlling microbial production rate by administering media with a microfluidic device, for a sufficient amount of time, and under conditions sufficient for the bacteria to produce nano-cellulose at a desired rate; and continuing the administering of the media until a target three-dimensional structure with a target thickness and target strength is formed which has a morphology defined by a network of multiple layers of interconnected biosynthetic cellulose. The disclosure further provides that porosity can be introduced to the structure by using porogens during the growth process, such as alginate or wax particles, which can be removed following biofabrication of the 3-D structure leaving behind pores of a desired shape and size. Such methods can be adapted for use with this invention to provide a plurality of BC scaffolds in wells of a microtiter plate.

Other methods of producing scaffolds are provided by International Application No. PCT/US2009/046407, filed Jun. 5, 2009 and entitled “Electromagnetic Controlled Biofabrication for Manufacturing of Mimetic Biocompatible Materials,” the disclosure of which is hereby incorporated by reference herein in its entirety. For example, the disclosure provides methods of producing a predetermined pattern of ordered biopolymers by applying an electromagnetic field to biopolymer extruding cells such that the cells extrude the biosynthetic cellulose in a desired manner to produce a scaffold of a particular morphology. Likewise, such methods can be adapted for use with the present invention as another representative means of obtaining controlled morphology biosynthetic cellulose for use in wells of a microtiter plate for bioassays. Indeed, any one or more of these applications may be combined to achieve particular desired results.

Additional advantages of nano-cellulose material for applications to support cell differentiation and production of tissue include, but are not limited to: the similarity of nano-cellulose fibrils to collagen fibrils mimicking the cellular extracellular matrix and providing topological cues for cell migration, attachment and differentiation; the porosity introduced in nano-cellulose networks may be designed specifically for each selected cell type to provide optimal production of tissue; the surface properties of nano-cellulose 3D scaffolds may provide minimal adsorption of proteins and small molecules; the chemical composition of cellulose may prevent the scaffold from degradation in living tissue, thereby preventing it from interfering with the products of the bioassay; the unique water binding capacity of nano-cellulose scaffold may provide a unique microenvironment which promotes better development of tissue; the unique ability to control microarchitecture of 3D nano-cellulose scaffold may make it possible to co-culture two or several cell types which enable cell cross talk and development of not only tissue but also of an organ; and the good biomechanical properties of nano-cellule 3D scaffold may make them suitable to use in a bioreactor to stimulate cells into tissue; and any combination thereof.

In certain embodiments, the present invention provides representative 3D scaffolding materials based on nano-cellulose that may control cell migration, proliferation and differentiation, thereby resulting in growth of living tissue with properties similar to native tissue. The design of scaffolding materials enables use of them with cells in bioreactors that may stimulate optimal tissue growth. The architecture and biomechanical properties of 3D porous nano-cellulose scaffolds as described in the present invention may make them suitable for stem cell differentiation and for growth of co-culture that may result in growth of tissues and artificial organs. In certain embodiments, the growth of living tissue may be performed in microtiter plates, making them easily available to a detector capable of sensing the biological activity of a selected drug. Detectors include, but are not limited to, robots, readers, and other on line analytical equipment that can function in a high throughput format. The microtiter plates used in the present invention include, but are not limited to, 96-well, 384-well, or 1536-well microtiter plates. In certain embodiments of the present invention, the 3D scaffold and cells to be grown into tissue or organs are placed in the microtiter plate at the same time. In other preferred embodiments, the 3D scaffold is placed in the microtiter plates prior to the addition of the cells. It may also be desired to grow the 3D scaffold and/or the cells directly in the microtiter wells. Each well comprising a 3D scaffold provides a unique environment that promotes the growth of tissue or organs in vitro. Once the tissue is grown, then each well can be used as a separate pharmacologic bioassay.

The three-dimensional (3-D) nano-cellulose based structures can be prepared in numerous ways. For example, scaffolds according to embodiments of the invention can comprise a network of multiple layers of biosynthetic cellulose forming a 3-D structure; wherein the network is fabricated in a non-mechanical manner; and wherein the 3-D structure has a density or tensile strength higher than nano-cellulose based 3-D structures formed from static-culture techniques or mechanical processes. Such scaffolds can be prepared in conjunction with methods for introducing controlled porosity into the scaffolds, which can be useful for various applications.

The present invention provides a tailor-made 3D architecture of microporous nano-cellulose scaffolds that provide a unique 3D microenvironment for optimal cell differentiation and production of extracellular matrix. In certain embodiments, the nano-cellulose may have a diameter in the range of about 10 nanometers and about 100 nanometers. The optimal diameter for the nano-cellulose will provide optimal nutrient and oxygen supply to cells. The porosity of the 3D scaffold can vary from the range of about 100 microns to about 500 microns. In certain embodiments, the pore architecture may vary depending on the type of cell being cultivated. One of ordinary skill in the art, with the benefit of this disclosure, will know the optimal diameter and porosity for use with each type of cell.

In an embodiment, the nano-cellulose binds water and forms a hydrogel. The hydrogel may be an ideal microenvironment for cell growth. The nano-cellulose described in the present invention may be produced by any known process. Certain processes for producing nano-cellulose include, but are not limited to, production by bacteria, electrospinning processes using cellulose derivatives that may be regenerated into cellulose, electrospinning processes using ionic liquids, foaming processes with or without porogens, freezing and freeze drying, and any combination thereof.

In certain embodiments of the present invention, tissues and organs are grown using the 3D scaffolds described herein. Examples of tissues and organs which can be grown using this invention include, but are not limited to skin, blood vessels, cardiovascular system, heart, cartilage, meniscus, bone, osteochondral tissue, joints, tendons, muscles, urinary tracts, bladder, neural networks, brain, artificial tumors and any combination thereof in so called coculture systems. In general terms 3D porous nano-cellulose scaffolds can support a variety of cell types including, but not limited to, primary and established cell lines. They are particularly suitable for stem cell support and differentiation.

In certain embodiments of the present invention microtiter plates comprising the 3D scaffold and the grown tissue or organ can be used to perform pharmacologic bioassays. Pharmacologic bioassays include, but are not limited to, drug screening assays, drug selection assays, drug development assays, drug toxicity assays, and any combination thereof. They can also be used to support basic research studies including, but not limited to, studies involving angiogenesis, cell migration and invasion, three-dimensional cell culture, neuronal cell culture, primary hepatocyte culture, culturing human embryonic stem (hES), and induced pluripotent stem (iPS) cells, bone marrow cells, osteoblasts, chondrocytes, fibrocytes, cancer cells, transfected cell lines, and any combination thereof.

To facilitate a better understanding of the present invention, the following examples of certain aspects of some embodiments are given. In no way should the following examples be read to limit, or define, the scope of the invention.

### EXAMPLE 1

### Development of Human Cartilage on Nano-Cellulose Scaffold

Nano-cellulose was prepared by fermentation of Gluconoacetobacter Xylinus using corn steep liquor medium. Wax particles size 300 microns were added during the fermentation process and removed by melting and repeated washing. An alternative route of making 3D porous scaffolds is homogenization of bacterial nano-cellulose using Ultratorax followed by freezing of dispersion at −80° C. followed by freeze drying at −50° C. for 24 hours. The resulting porous 3D nano-cellulose scaffold was placed in wells in 96 microtiter plates, sterilized and seeded with human articular chondrocytes. To study formation of cartilage, cells isolated from three patients were expanded and seeded onto 3D porous nano-cellulose scaffolds. Dulbecco's modified eagle's media (DMEM)/F12 (Invitrogen, Grand Island, N.Y.) supplemented with L-ascorbic acid (0.025 mg/mL), gentamicin sulfate (50 mg/L), amphoterricin B (250 mg), L-glutamine (2 mM), and 10% human serum. After incubation over night, cells were seeded onto 3D porous nano-cellulose scaffolds and chondrogenic culturing media DMEM high glucose supplemented with ITS linoleic acid (5.0 mg/mL), human serum albumin (1.0 mg/mL), TGF-b1 (10 ng/mL), dexamethasone, ascorbic acid (14 microg/mL), and penicillinstreptomycin. Cultivation was performed at 37° C. and 5% CO₂ under static conditions in incubator. The media were changed every third day and cultivation was carried out for 14 days, 21 days, and 28 days and scaffold-cell constructs were analyzed with histological staining for ECM components. The amount of DNA in scaffolds was analyzed biochemically by rinsing with PBS and DNA extracted by digesting scaffolds with Papain solution; 0.3 mg Papain/mL sodium phosphate buffer (30 mM) with 1 mM EDTA and 2 mM dithiothreitol at 60° C. for 24 h. The amount of DNA was measured spectrophotometrically with Hoechst 33258 solution (about 0.2 mg/L). Histological staining for glycosaminoglycans (GAGS) was used to study ECM production by chondrocytes. Scaffolds-cell constructs were pretreated and stained with Alcian blue van Gieson solutions. The analysis of DNA showed that cell proliferated in 3D porous nano-cellulose scaffold and proliferation rate increased after 14 days cultivation. Histological analysis showed that human chondrocytes produced in 3D porous nano-cellulose scaffold ECM were characteristic for human cartilage.

### EXAMPLE 2

### Growth of Co-Culture of Endothelial and Smooth Muscle Cells—Model of Arteries and Blood Vessels to Study Arteriosclerosis and Plaque Formation

3D nano-cellulose scaffolds were designed to mimic (e.g., represent, copy, be similar to, be characterized by, etc.) vascular tissues. Channels were produced by inserting optical fibers with diameter of 500 micron and surrounded by wax particles of diameter 200 microns in bacterial cellulose fermentation process. 3D nano-cellulose scaffold produced this way was purified and sterilized. Scaffold was then placed in the bottom of the titer microplate. Endothelial cells (HSVECs) and smooth muscle cells were isolated from non-diseased human saphenous veins, by-products of coronary bypass surgery. Cells were isolated using an enzymatic technique using a solution of 0.1% collagenase type I in Phosphate Buffered Saline. Endothelial cells were then seeded in channels of nano-cellulose scaffold and smooth muscle cells were seeded in a porous part of the scaffold prepared by using wax porogens. Cells were cultured at 37° C. in a humidified incubator with 5% CO₂. Dulbecco Modified Eagle Medium with 10% of fetal calf serum and 10 ng/mL platelet derived growth factor was used as medium. Both cell types were cultured for 2 weeks. Endothelial cells had the characteristic morphological cobblestone pattern, were positive for antibodies against PECAM-1 and von Willebrand factor. After two weeks of cultivation endothelial cells formed confluent layer in channels mimicking vascular tissues. Smooth muscle cells were producing extracellular matrix as shown by collagen detection. Thus, a good model for vascular tissue was cultivated onto 3D microporous nano-cellulose scaffold in microtiter plates.

### EXAMPLE 3

### Human Mesenchymal Stem Cell Differentiation in 3D Porous Nano-Cellulose

3D microporous nano-cellulose scaffolds with porosity of 300 microns produced using wax porogens were pretreated with anionic polysaccharides such as carboxymethylcellulose followed by treatment with simulated body fluid to produce biomimetic coating consisting of hydroxyapatite. Such scaffolds were seeded with human mesenchymal stem cells. The differentiation media (growth media supplemented with 0.13 mM ascorbic acid 2-phosphate, 2 mM β-glycerophosphate and 10 nM dexamethasone) was used. Cells were cultivated in an incubator at 37° C., 5% CO2 and 95% relative humidity. The culture medium was changed every third day. The proliferation was studied using MTS assay and results showed that the cells proliferated. Samples at 7, 14 and 21 days were analyzed with Alkaline Phosphatase ELISA Assay Kit assay. Results showed that human mesenchymal stem cells have differentiated into osteoblasts after 21 days cultivation as shown by producing extracellular matrix characteristic for osteoblast cells.

As shown and described in this specification, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Indeed, tt will be apparent to those skilled in the art that various modifications and variations can be made in the practice of the present invention without departing from the scope or spirit of the invention. Furthermore, no limitations are intended to the details of construction or design herein shown, other than as described in the claims below.

The present invention has been described with reference to particular embodiments having various features. One skilled in the art will recognize that these features may be used singularly or in any combination based on the requirements and specifications of a given application or design. Other embodiments of the invention will be apparent to those skilled in the art from consideration of the specification and practice of the invention. Where a range of values is provided in this specification, each value between the upper and lower limits of that range is also specifically disclosed. The upper and lower limits of these smaller ranges may independently be included or excluded in the range as well. All numbers and ranges disclosed above may vary by some amount. As used in this specification, the singular forms “a,” “an,” and “the” include plural referents unless the context clearly dictates otherwise. While compositions and methods are described in terms of “comprising,” “containing,” or “including” various components or steps, the compositions and methods can also “consist essentially of” or “consist of” the various components and steps. It is intended that the specification and examples be considered as exemplary in nature and that variations that do not depart from the essence of the invention are intended to be within the scope of the invention.

Further, the references cited in this disclosure are incorporated by reference herein in their entireties. If there is any conflict in the usages of a word or term in this specification and one or more or other documents that may be incorporated herein by reference, the definitions that are consistent with this specification should be adopted.

1. A device for high throughput bioassays comprising:
a structural support having a plurality of recesses; and
a plurality of non-biodegradable nano-cellulose scaffolds, each disposed in a recess of the support and having a selected 3D porous architecture.

2. The device of claim 1, wherein the nano-cellulose is fabricated in a non-mechanical manner to provide a network of biosynthetic cellulose, electrospun fibers or plant derived nanofibrilated cellulose.

3. The device of claim 1, wherein the 3D porous architecture is selected to provide for cell differentiation and production of extracellular matrix.

4. The device of claim 3, further comprising living cells.

5. A high throughput system for drug evaluation comprising:
a drug having a pharmacological activity of interest; and
a microtiter plate comprising a plurality of living tissues or organs, each engineered on a 3D non-biodegradable, inert, nano-cellulose scaffold support, operably configured to allow for in vitro testing of the drug on a plurality of tissues or organs simultaneously.

6. The system of claim 5, wherein the scaffold supports have a selected 3D porous architecture operably configured to represent a native tissue or organ.

7. A kit for a pharmacologic bioassay platform comprising: a microtiter plate comprising a plurality of 3D non-biodegradable, inert, nano-cellulose scaffold supports, each having a selected thickness and microporosity;
living cells of a selected tissue type; and
media for growing the cells on the scaffolds.

8-12. (canceled)
